(19) United States
(12) Patent Application Publication
(10) Pub. No.: US 2004/0231016 A1
(43) Pub. Date: Nov. 18, 2004

(54) Efficient gene silencing in plants using short dsRNA sequences

(75) Inventors: Ming Bo Wang, Canberra (AU); Christopher Andrew Helliwell, O'Connor (AU); Peter Michael Waterhouse, O'Connor (AU)

Correspondence Address: BURNS DOANE SWECKER & MATHIS L L P, POST OFFICE BOX 1404, ALEXANDRIA, VA, 22313-1404, US

(73) Assignee: Commonwealth Scientific and Industrial Research Organization

(21) Appl. No.: 10/780,638

(22) Filed: Feb. 19, 2004

Related U.S. Application Data
(60) Provisional application No. 60/447,711, filed on Feb. 19, 2003.

(51) Int. Cl. A01H 1/00; C12N 15/82

(52) U.S. Cl. 800/278

(57) ABSTRACT

Methods and means are provided to increase the efficiency of gene silencing when using dsRNA sequences which have a stem length shorter than about 200 base pairs by providing chimeric genes encoding such dsRNA sequences with a promoter recognized by DNA dependent RNA polymerase III comprising all cis-acting promoter elements which interact with DNA dependent RNA polymerase III.

### FIELD OF THE INVENTION

The current invention relates generally to the field of genetic modification of plants, more particularly to the use of short double stranded (dsRNA) sequences to deliberately silence the expression of one or more genes in plant cells and plants. Methods and means are provided to increase the efficiency of gene silencing when using dsRNA sequences which have a stem length shorter than about 200 base pairs.

### BACKGROUND

The mechanism of posttranscriptional silencing of gene expression in plants and animals triggered by target-gene specific dsRNA, provided either exogenously or endogenously through transcription of dsRNA encoding chimeric genes, has recently become the subject of numerous studies. Since the initial description of this phenomenon in animals and plants (Fire et al., 1998; Hamilton et al., 1998; Waterhouse et al., 1998), it has become clear that the dsRNA is processed by an RNAse with a preference for dsRNA (such as DICER in Drosophila) into short, approximately 21 nucleotide long RNA molecules that are used as guide sequences, providing sequence-specificity to a complex capable of degrading specific mRNA molecules.

The high specificity and efficiency of gene silencing initiated by dsRNA that is homologous to the gene to be silenced rapidly turned this methodology into the preferred tool to generate eukaryotic organisms wherein expression of one or more specific transcribed nucleotide sequences is reduced or inactivated. Such reduction or inactivation of the expression of a gene of interest may be achieved with a goal to produce eukaryotic organisms with a preferred phenotype (see e.g. WO 02/029028, wherein Brassica plants are generated which develop sepals instead petals using dsRNA technology). Reduction or inactivation of expression of transcribed sequences also plays an important role in experimental studies trying to allocate a function to the wealth of nucleotide sequences which have become available through various genome sequencing programs.

Particularly for the latter, it may be advantageous to use short dsRNA sequences, since such oligonucleotides may conveniently be generated in vitro. In higher animals, the use of short dsRNA molecules is preferred in view of the fact that larger dsRNA molecules seem to trigger interferon responses (Elbashir et al. 2001).

Up to now, the production of inhibitory RNA (used herein to describe antisense RNA, sense RNA and dsRNA) inside the cells of eukaryotic organisms, mostly occurs through the action of DNA dependent RNA polymerase II (PolII) recognizing the common PolII type promoters.

Antisense RNA production through the action of RNA polymerase III in plants has been documented.

Bourque and Folk (1992) described suppression of the expression of a CAT gene, transiently delivered to plant cells, by co-electroporation with a DNA comprising inverted sequences of the chloramphenicol actetyltransferase reporter gene, fused to a soybean tRNA^{met} gene lacking a terminator, such that the tRNA^{met} sequences caused the transcription of CAT antisense sequences by RNA polymerase III.

U.S. Pat. No. 5,354,854 describes an expression system and method to use the same in plants to suppress gene expression, the system including a constitutive promoter element from a tRNA gene and an antisense strand DNA fused to the promoter element for being co-transcribed with the promoter element by an RNA polymerase III to suppress expression of a gene.

Yukawa et al. 2002 described antisense RNA sequences targeted against conserved structural elements or domains in the RNAs of potato spindle tuber viroid, hop latent viroid and potato virus S which were embedded in the anticodon region or a Nicotiana tRNA^{tyr} gene or near the 3′ end of an Arabidopsis 7 SL RNA gene, and demonstrated in vitro transcription of such chimeric genes in a homologous plant extract.

EP 0 387 775 describes and claims a DNA molecule, optionally occurring in multiple copies, containing sections of a gene transcribed by polymerase III and a DNA sequence encoding for an inhibiting RNA molecule, characterized in that it contains the transcription units of a tRNA gene necessary for transcription by polymerase III, including the sequence which determine the secondary structure of the tRNA, and that the DNA sequence coding for the inhibiting RNA molecule is arranged inside the DNA molecule in such a way that the inhibiting RNA molecule is a part of the transcript.

Expression of small interfering RNAs in mammalian cells has recently been well documented. Paddison et al. 2002; Sook Lee et al. 2002; Miyagishi et al. 2002, Sui et al. 2002; Brummelkamp et al, 2002 and Paul et al. 2002, all describe the expression of small interfering RNA in human or mammalian cells using RNA polymerase III specific promoters derived from either H1-RNA or U6 snRNA.

U.S. Pat. No. 6,146,886 describes and claims a transcribed non-naturally occurring RNA molecule comprising a desired RNA portion, wherein said non-naturally occurring RNA molecule comprises an intramolecular stem formed by base-pairing interactions between a 3′ region and 5′ complementary nucleotides in said RNA, wherein said intramolecular stem comprises at least 8 base pairs; wherein said desired RNA portion is selected from the group consisting of antisense RNA, decoy RNA, enzymatic RNA, agonist RNA and antagonist RNA, wherein said RNA molecule is transcribed by a type 2 RNA polymerase III promoter system.

The prior art remains however deficient in providing methods for highly efficient expression of small interfering dsRNAs in plant cells. This problem has been solved as hereinafter described.

### SUMMARY OF THE INVENTION

The invention provides methods for reducing the expression of a gene of interest in a plant cell, comprising the following steps:

1) providing a chimeric gene to the plant cell, the chimeric gene comprising the following operably linked DNA fragments:

a) a promoter recognized by a DNA dependent RNA polymerase III of the plant cell characterized in that the promoter is a promoter of type III (type 3) comprising all cis-acting promoter elements which interact with the DNA dependent RNA polymerase III, for example a type 3 POLIII promoter selected from the promoter of a gene encoding U6snRNA, the promoter of a gene encoding U3snRNA, the promoter of a gene encoding 7SL RNA, more preferably a promoter comprising the nucleotide sequence of promoter is selected from the nucleotide sequences of SEQ ID No. 1, SEQ ID No. 2, SEQ ID No. 3, SEQ ID No. 4, SEQ ID No. 5, SEQ ID No. 6, SEQ ID No. 7 or SEQ ID No. 8;

b) a DNA fragment which, when transcribed, yields an RNA molecule, the RNA molecule comprising a sense and antisense nucleotide sequence,

i) the sense nucleotide sequence comprising about 19 contiguous nucleotides having about 90 to about 100% sequence identity to a nucleotide sequence of about 19 contiguous nucleotide sequences from the RNA transcribed from the gene of interest;

ii) the antisense nucleotide sequence comprising about 19 contiguous nucleotides having about 90 to 100% sequence identity to the complement of a nucleotide sequence of about 19 contiguous nucleotide sequence of the sense sequence;

wherein the sense and antisense nucleotide sequence are capable of forming a double stranded RNA of about 19 to about 200 nucleotides in length; and

c) an oligo dT stretch comprising at least 4 consecutive T-residues; and

2) identifying plant cells wherein the expression of the gene of interest is reduced when compared to the expression of the gene of interest in plant cells which do not comprise the chimeric gene.

The invention further provides a chimeric gene comprising the following operably linked DNA fragments:

1) a promoter recognized by a DNA dependent RNA polymerase III of the plant cell characterized in that the promoter is a promoter of type III comprising all cis-acting promoter elements which interact with the DNA dependent RNA polymerase III, for example a type 3 POLIII promoters selected from the promoter of a gene encoding U6snRNA, the promoter of a gene encoding U3snRNA, the promoter of a gene encoding 7SL RNA, more preferably a promoter comprising the nucleotide sequence of promoter is selected from the nucleotide sequences of SEQ ID No. 1, SEQ ID No. 2, SEQ ID No. 3, SEQ ID No. 4, SEQ ID No. 5, SEQ ID No. 6, SEQ ID No. 7 or SEQ ID No. 8;

2) a DNA fragment which, when transcribed, yields an RNA molecule, the RNA molecule comprising a sense and antisense nucleotide sequence,

a) the sense nucleotide sequence comprising about 19 contiguous nucleotides having about 90 to about 100% sequence identity to a nucleotide sequence of about 19 contiguous nucleotide sequences from the RNA transcribed from a gene of interest in a plant cell;

b) the antisense nucleotide sequence comprising about 19 contiguous nucleotides having about 90 to 100% sequence identity to the complement of a nucleotide sequence of about 19 contiguous nucleotide sequence of the sense sequence;

wherein the sense and antisense nucleotide sequence are capable of forming a double stranded RNA of about 19 to about 200 nucleotides in length; and

c) an oligo dT stretch comprising at least 4 consecutive T-residues.

The invention further provides plant cell and plants comprising the above mentioned chimeric genes

### BRIEF DESCRIPTION OF THE FIGURE

FIG. 1 outlines schematically a convenient cloning strategy for creating and handling a coding region encoding short dsRNA sequences.

### DETAILED DESCRIPTION

The current invention is based on the observation that chimeric genes encoding short dsRNA molecules, preferably ranging between about 20 base pairs (bp) and about 100 bp under control of type 3 promoters recognized by RNA polymerase III, resulted in more efficient gene silencing than similar constructs driven by the strong constitutive RNA Polymerase II promoter CaMV 35S.

These type 3 promoters have the additional advantage that all required cis-acting elements of the promoter are located in the region upstream of the transcribed region, in contrast to type 2 promoters recognized by RNA polymerase III, which had been used in the prior art to direct expression of antisense RNA.

Thus, in a first embodiment, the current invention relates to a method for reducing the expression of a gene of interest in a plant cell, comprising the following steps:

1) providing a chimeric gene to the plant cell, the chimeric gene comprising the following operably linked DNA fragments:

a) a promoter recognized by a DNA dependent RNA polymerase III of the plant cell whereby the promoter is a promoter of type 3 comprising all cis-acting promoter elements which interact with DNA dependent RNA polymerase III;

b) a DNA fragment which, when transcribed, yields an RNA molecule, the RNA molecule comprising a sense and antisense nucleotide sequence, and wherein

i) the sense nucleotide sequence comprises about 19 contiguous nucleotides having about 90 to about 100% sequence identity to a nucleotide sequence of about 19 contiguous nucleotide sequences from the RNA transcribed from the gene of interest;

ii) the antisense nucleotide sequence comprising about 19 contiguous nucleotides having about 90 to 100% sequence identity to the complement of a nucleotide sequence of about 19 contiguous nucleotide sequence of the sense sequence;

wherein the sense and antisense nucleotide sequence are capable of forming a double stranded RNA of about 19 to about 200 nucleotides in length; and

c) an oligo dT stretch comprising at least 4 consecutive T-residues; and

2) identifying plant cells wherein the expression of the gene of interest is reduced when compared to the expression of the gene of interest in plant cells which do not comprise the chimeric gene.

As used herein, “a promoter recognized by the DNA dependent RNA polymerase III” is a promoter which directs transcription of the associated DNA region through the polymerase action of RNA polymerase III. These include genes encoding 5S RNA, tRNA, 7SL RNA, U6 snRNA and a few other small stable RNAs, many involved in RNA processing. Most of the promoters used by Pol III require sequence elements downstream of +1, within the transcribed region. A minority of pol III templates however, lack any requirement for intragenic promoter elements. These are referred to as type 3 promoters. In other words, “type 3 Pol III promoters” are those promoters which are recognized by RNA polymerase III and contain all cis-acting elements, interacting with the RNA polymerase III upstream of the region normally transcribed by RNA polymerase III. Such type 3 Pol III promoters can thus easily be combined in a chimeric gene with a heterologous region, the transcription of which is desired, such as the dsRNA coding regions of the current invention.

Typically, type 3 Pol III promoters contain a TATA box (located between −25 and −30 in Human U6 snRNA gene) and a Proximal Sequence element (PSE; located between −47 and −66 in Human U6 snRNA). They may also contain a Distal Sequence Element (DSE; located between −214 and −244 in Human U6 snRNA).

Type 3 Pol III promoters can be found e.g. associated with the genes encoding 7SL RNA, U3 snRNA and U6 snRNA. Such sequences have been isolated from Arabidopsis, rice and tomato and representative sequences of such promoters are represented in the sequence listing under the entries SEQ ID No 1-8.

Other nucleotide sequences for type 3 Pol III promoters can be found in nucleotide sequence databases under the entries for the A. thaliana gene AT7SL-1 for 7SL RNA (X72228), A. thaliana gene AT7SL-2 for 7SL RNA (X72229), A. thaliana gene AT7SL-3 for 7SL RNA (AJ290403), Humulus lupulus H17SL-1 gene (AJ236706), Humulus lupulus H17SL-2 gene (AJ236704), Humulus lupulus H17SL-3 gene (AJ236705), Humulus lupulus H17SL-4 gene (AJ236703), A. thaliana U6-1 snRNA gene (X52527), A. thaliana U6-26 snRNA gene (X52528), A. thaliana U6-29 snRNA gene (X52529), A. thaliana U6-1 snRNA gene (X52527), Zea mays U3 snRNA gene (Z29641), Solanum tuberosum U6 snRNA gene (Z17301; X 60506; S83742), Tomato U6 smal nuclear RNA gene (X51447), A. thaliana U3C snRNA gene (X52630), A. thaliana U3B snRNA gene (X52629), Oryza sativa U3 snRNA promoter (X79685), Tomato U3 smal nuclear RNA gene (×14411), Triticum aestivum U3 snRNA gene (X63065), Triticum aestivum U6 snRNA gene (X63066).

It goes without saying that variant type 3 Pol III promoters may be isolated from other varieties of tomato, rice or Arabidopsis, or from other plant species without little experimentation. For example, libraries of genomic clones from such plants may be isolated using U6 snRNA, U3 snRNA or 7SL RNA coding sequences (such as the coding sequences of any of the above mentioned sequences identified by their accession number and additionally the Vicia faba U6snRNA coding sequence (X04788), the maize DNA for U6 snRNA (X52315) or the maize DNA for 7SL RNA (X14661)) as a probe, and the upstream sequences, preferably the about 300 to 400 bp upstream of the transcribed regions may be isolated and used as type 3 Pol III promoters. Alternatively, PCR based techniques such as inverse-PCR or TAIL®-PCR may be used to isolate the genomic sequences including the promoter sequences adjacent to known transcribed regions. Moreover, any of the type 3 PolIII promoter sequences attached or of the above mentioned promoter sequences, identified by their accession numbers, may be used as probes under stringent hybridization conditions or as source of information to generate PCR primers to isolate the corresponding promoter sequences from other varieties or plant species.

“Stringent hybridization conditions” as used herein mean that hybridization will generally occur if there is at least 95% (or at least 97%) sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

Although the type 3 Pol III promoters have no requirement for cis-acting elements located with the transcribed region, it is clear that sequences normally located downstream of the transcription initiation site may nevertheless be included in the chimeric constructs of the invention.

It has also been observed that type 3 Pol III promoters originally isolated from monocotyledonous plants can be used to good effect in both dicotyledonous and monocotyledonous plant cells and plants, whereas type 3 Pol III promoters originally isolated from dicotyledonous plants can only be efficiently used in dicotyledonous plant cells and plants. Moreover, the most efficient gene silencing has been obtained when chimeric genes were used comprising a type 3 Pol III promoter derived from the same or closely related species.

As used herein, a “gene of interest” may be any nucleic acid of interest, which is transcribed (or replicated) into an RNA molecule, and which is prone to post-transcriptional RNA degradation. These include but are not limited to transgenes, endogenous genes and transcribed viral sequences. It will also be immediately apparent that for the methods of the invention, it is not required to have knowledge of the nucleotide sequence of the gene of interest. Indeed, it may be possible to directly derive small fragments and operably link them in inverted repeat orientation, under control of a type 3 Pol III promoter

As indicated above, the transcribed DNA region should be capable of encoding an RNA molecule comprising a sense and antisense nucleotide region, whereby the sense nucleotide sequence comprises about 19 contiguous nucleotides having about 90 to about 100% sequence identity to a nucleotide sequence of about 19 contiguous nucleotide sequences from the RNA transcribed from the gene of interest and whereby the antisense nucleotide sequence comprising about 19 contiguous nucleotides having about 90 to 100% sequence identity to the complement of a nucleotide sequence of about 19 contiguous nucleotide sequence of the sense sequence. The sense and antisense nucleotide sequence should be capable of forming a double stranded RNA of about 19 to about 200 nucleotides, alternatively about 21 to about 90 or 100 nucleotides, or alternatively about 40 to about 50 nucleotides in length. However, the length of the dsRNA stem may also be about 30, about 60, about 70 or about 80 nucleotides in length. It will be clear that where the dsRNA region is larger than 19 nucleotides, there is only a requirement that there is at least one double stranded region of about 19 nucleotides (whereby there can be about one mismatch between the sense and antisense region) the sense strand of which is “identical” (allowing for one mismatch) with 19 consecutive nucleotides of the target nucleic acid or gene of interest.

For the purpose of this invention, the “sequence identity” of two related nucleotide sequences, expressed as a percentage, refers to the number of positions in the two optimally aligned sequences which have identical residues (×100) divided by the number of positions compared. A gap (i.e., a position in an alignment where a residue is present in one sequence but not in the other) is regarded as a position with non-identical residues. The alignment of the two sequences is performed by the Needleman and Wunsch algorithm (Needleman and Wunsch 1970). Computer-assisted sequence alignment, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

The transcribed DNA region may comprise a stretch of nucleotides ranging from 3 to about 100 nucleotides or alternatively from about 6 to about 40 nucleotides, which are located between the sense and antisense encoding nucleotide region, and which are not related to the nucleotide sequence of the target gene (a so-called spacer region).

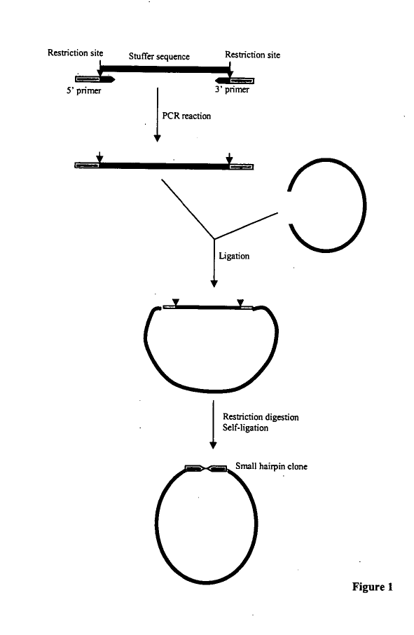

The chimeric genes of the current invention, comprising a transcribed DNA region with short antisense and sense fragments may conveniently be constructed using a stuffer DNA sequence between the short antisense and sense fragments during the cloning procedures, which may thereafter be removed. To that end, the stuffer segment may be equipped with restriction enzymes recognitions sites, such as rare-cutting restriction enzymes for the easy removal of the stuffer sequence and re-ligation (self-ligation) of the cloning vector, whereby the short sense and antisense region are now brought in vicinity of each other. As outlined in FIG. 1, a DNA fragment comprising a short sense sequence, a short, antisense sequence complementary to the sense sequence, and a stuffer DNA sequence may be conveniently construct by PCR amplification using oligonucleotide primers comprising the sense or antisense sequence and a sequence corresponding to part of the stuffer DNA sequence.

The above mentioned “oligo dT stretch” is a stretch of consecutive T-residues which serve as a terminator for the RNA polymerase III activity. It should comprise at least 4 T-residues, but obviously may contain more T-residues.

Chimeric genes according to the invention may be provided to plant cells by introduction into plant cells using any means of DNA transformation available in the art, including but not limited to Agrobacterium-mediated transformation, microprojectile bombardment, direct DNA uptake into protoplasts or plant tissues (by electroporation, PEG-mediated uptake, etc.) and may result in transiently or stably transformed plant cells. The chimeric genes may also be provided to the plant cells using viral vectors, capable of replicating in plant cells. Chimeric genes may also be provided to plant cells by crossing parental plants, at least one of which comprises a chimeric gene according to the invention.

As used herein, “reducing the expression of a gene of interest” refers to the comparison of the expression of the gene of interest in the plant cell in the presence of the dsRNA or chimeric genes of the invention, to the expression of the gene of interest in the absence of the dsRNA or chimeric genes of the invention. The expression in the presence of the chimeric RNA of the invention should thus be lower than the expression in absence thereof, e.g. be only about 75% or 50% or 10% or about 5% of the expression in absence of the chimeric RNA. The expression may be completely inhibited for all practical purposes by the presence of the chimeric RNA or the chimeric gene encoding such RNA.

A reduction of expression of a gene of interest may be measured as a reduction in transcription of (part of) that gene, a reduction in translation of (part of) that gene or a reduction in the effect the presence of the transcribed RNA(s) or translated polypeptide(s) have on the plant cell or the plant, and will ultimately lead to altered phenotypic traits. It is clear that the reduction in expression of a gene of interest may be accompanied by or correlated to an increase in expression of another gene. Although the main effect of dsRNA is the post transcriptional degradation of specific RNAs, effects of dsRNA on the transcription process have been documented. Such additional effects will also contribute to the reduction of expression of a gene of interest mediated by dsRNA.

Other embodiments of the invention relate to the chimeric genes as herein described, as well as to plants, plant cells, plant tissues or seeds comprising the chimeric genes of the invention.

It is also an object of the invention to provide plant cells and plants containing the chimeric genes according to the invention. Gametes, seeds, embryos, either zygotic or somatic, progeny or hybrids of plants comprising the chimeric genes of the present invention, which are produced by traditional breeding methods, are also included within the scope of the present invention.

The methods and means described herein are believed to be suitable for all plant cells and plants, both dicotyledonous and monocotyledonous plant cells and plants including but not limited to cotton, Brassica vegetables, oilseed rape, wheat, corn or maize, barley, sunflowers, rice, oats, sugarcane, soybean, vegetables (including chicory, lettuce, tomato), tobacco, potato, sugarbeet, papaya, pineapple, mango, Arabidopsis thaliana, but also plants used in horticulture, floriculture or forestry.

The following non-limiting Examples describe the construction of chimeric genes for the reduction of the expression of a gene of interest in a plant cell by small dsRNA and the use of such genes.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Throughout the description and Examples, reference is made to the following sequences:

SEQ ID No. 1: sequence of the promoter of the 7SL-2 gene of Arabidopsis thaliana var. Landsberg erecta, followed by a unique restriction site in front of an oligo dT stretch.

SEQ ID No. 2: sequence of the promoter of the 7SL-2 gene of Arabidopsis thaliana var. Landsberg erecta including 86 bases downstream of the transcription initiation site, followed by a unique restriction site in front of an oligo dT stretch.

SEQ ID No. 3: sequence of the promoter of the U3B snRNA of Arabidopsis thaliana var. Landsberg erecta, followed by a unique restriction site in front of an oligo dT stretch.

SEQ ID No. 4: sequence of the promoter of the U3B snRNA gene of Arabidopsis thaliana var. Landsberg erecta including 136 bases downstream of the transcription initiation site, followed by a unique restriction site in front of an oligo dT stretch.

SEQ ID No. 5: sequence of the promoter of the U6-26 snRNA gene of Arabidopsis thaliana var. Landsberg erecta including 3 bases downstream of the transcription initiation site, followed by a unique restriction site in front of an oligo dT stretch.

SEQ ID No. 6: sequence of the promoter of the U6-26 snRNA gene of Arabidopsis thaliana var. Landsberg erecta including 20 bases downstream of the transcription initiation site, followed by a unique restriction site in front of an oligo dT stretch.

SEQ ID No. 7: sequence of the promoter of the U3 snRNA of rice (Oryza sativa Indica IR36), followed by a unique restriction site in front of an oligo dT stretch.

SEQ ID No. 8: sequence of the promoter of the U3 snRNA of tomato (a garden variety with small gourd-shaped yellow fruit), followed by a unique restriction site in front of an oligo dT stretch.

SEQ ID No. 9: sequence of the dsRNA encoding region of 94 bp for silencing expression of the GUS gene (GUShp94).

SEQ ID No. 10: sequence of the dsRNA encoding region of 41 bp for silencing expression of the GUS gene (GUShp41).

SEQ ID No. 11: sequence of the dsRNA encoding region of 21 bp for silencing expression of the GUS gene (GUShp21).

SEQ ID No. 12: sequence of the dsRNA encoding region of 42 bp for silencing expression of the PHYB gene, derived from the 5′ end of PHYB (PHYB5hp42)-upper strand.

SEQ ID No. 13: sequence of the dsRNA encoding region of 42 bp for silencing expression of the PHYB gene, derived from the 5′ end of PHYB (PHYB5hp42)-lower strand.

SEQ ID No. 14: sequence of the dsRNA encoding region of 21 bp for silencing expression of the PHYB gene, derived from the 5′ end of PHYB (PHYB5hp21)-upper strand.

SEQ ID No. 15: sequence of the dsRNA encoding region of 21 bp for silencing expression of the PHYB gene, derived from the 5′ end of PHYB (PHYB5hp21)-lower strand.

SEQ ID No. 16: sequence of the dsRNA encoding region of 42 bp for silencing expression of the PHYB gene, derived from the center of PHYB (PHYBChp42)-upper strand.

SEQ ID No. 17: sequence of the dsRNA encoding region of 42 bp for silencing expression of the PHYB gene, derived from the center of PHYB (PHYBChp42)-lower strand.

SEQ ID No. 18: sequence of the dsRNA encoding region of 21 bp for silencing expression of the PHYB gene, derived from the center of PHYB (PHYBChp21)-upper strand.

SEQ ID No. 19: sequence of the dsRNA encoding region of 21 bp for silencing expression of the PHYB gene, derived from the center of PHYB (PHYBChp21)-lower strand.

SEQ ID No. 20: sequence of the dsRNA encoding region of 42 bp for silencing expression of the PHYB gene, derived from the 3′ end of PHYB (PHYB3hp42)-upper strand.

SEQ ID No. 21: sequence of the dsRNA encoding region of 42 bp for silencing expression of the PHYB gene, derived from the 3′ end of PHYB (PHYB3hp42)-lower strand.

SEQ ID No. 22: sequence of the dsRNA encoding region of 21 bp for silencing expression of the PHYB gene, derived from the 3′ end of PHYB (PHYB3hp21)-upper strand.

SEQ ID No. 23: sequence of the dsRNA encoding region of 21 bp for silencing expression of the PHYB gene, derived from the 3′ end of PHYB (PHYB3hp21)-lower strand.

SEQ ID No. 24: sequence of the dsRNA encoding region of 42 bp for silencing expression of the PDS gene (PDS42)-upper strand.

SEQ ID No. 25: sequence of the dsRNA encoding region of 42 bp for silencing expression of the PDS gene (PDS42)-lower strand.

SEQ ID No. 26: sequence of the dsRNA encoding region of 21 bp for silencing expression of the PDS gene (PDS21)-upper strand.

SEQ ID No. 27: sequence of the dsRNA encoding region of 21 bp for silencing expression of the PDS gene (PDS21)-lower strand.

SEQ ID No. 28: sequence of a dsRNA encoding region of 42 bp for silencing expression of a GUS gene (GUS-A)

SEQ ID No. 29: sequence of a dsRNA encoding region of 42 bp for silencing expression of a GUS gene (GUS-B).

SEQ ID No. 30: sequence of a dsRNA encoding region of 42 bp for silencing expression of a GUS gene (GUS-C).

SEQ ID No. 31: sequence of a dsRNA encoding region of 42 bp for silencing expression of EIN (EIN-A).

SEQ ID No. 32: sequence of a dsRNA encoding region of 42 bp for silencing expression of EIN (EIN-B).

SEQ ID No. 33: sequence of a dsRNA encoding region of 42 bp for silencing expression of EIN (EIN-C).

### EXAMPLES

### Example 1

### Construction of Type 3 Pol III Promoter-OligodT Stretch Cassettes

Type 3 Pol III promoters were isolated from Arabidopsis, rice or tomato 7SL, U3snRNA or U6snRNA genes using PCR amplification, designed in such a way that

1) the resulting fragments were flanked by restriction enzyme recognition sites not present within the amplified fragment;

2) the promoter fragments were followed by a unique restriction site (SalI, XhoI or PvuI), followed by

3) a poly(T) sequence (with 7-9 T residues) as Pol III terminator.

In some of the cloned promoter fragments, additional sequences of the coding region downstream of the transcription initiation site were included to investigate the possible effect of conserved motifs in the coding region of the small RNAs on transcription and/or gene silencing. The resulting fragments (represented in SEQ IDs No 1 to 8) were cloned in intermediate cloning vectors (see Table 1). Sense, antisense or inverted repeat sequences can readily be inserted in the unique restriction site between the type 3 Pol III promoters and the polyT stretch.

TABLE 1
Cloned PoIIII promoter-terminator cassettes cloned*

| | | | | Name of |
|---|---|---|---|---|
| | Cloned | Size | | intermediate |
| Small RNAs | promoters | (bp)*** | Plant species | plasmid |
| 7SL-2 | At7SL-P | 343 | Arabidopsis | pMBW444 |
| | At7SL+86** | 432 | (L.er) | pMBW445 |
| U3B | AtU3B-P | 334 | Arabidopsis | pMBW442 |
| | AtU3B+136 | 467 | (L.er) | pMBW426 |
| U6-26 | AtU6+3 | | Arabidopsis | PWGEM.U6+3 |
| | AtU6+20 | | (L.er) | PWGEM.U3+20 |
| U3 | OsU3-P | 407 | Rice (Oryza | pMBW446-LW |
| | | | sativa indica | |
| | | | IR36) | |
| U3 | TomU3-P | 443 | Tomato (a | pMBW443-LW |
| | | | garden | |
| | | | variety with | |
| | | | small gourd- | |
| | | | shaped | |
| | | | yellow fruit) | |

**This number represents the sequence from the coding region of the small RNA gene.
***The sizes given include the restriction sites and the oligo (dT)s added to the PCR primers.

### Example 2

### Testing of the PolIII Promoters in Gene Silencing Constructs Against a GUS Reporter Gene (Nicotiana tabacum).

To test these PolIII promoters for silencing, a GUS inverted-repeat sequence (SEQ ID No 9) was synthesized, which consists of 186 bp sense sequence of GUS (nt. 690-875 of GUS coding sequence) fused at the 3′ end with an antisense version of the first 94 bp in the 186 bp fragment (nt. 690-783 of GUS coding sequence). This i/r sequence is flanked by two SalI sites and two PvuI sites, and can therefore be cloned into the PolIII promoter vectors as a SalI or PvuI fragment. Constructs were prepared with all the PolIII promoters described in Table 1 using the i/rGUS sequence (GUShp94) (see Table 2). In addition to the GUShp94 sequence, constructs were also prepared with the AtU3B+136 promoter (SEQ ID No 4.) and the CaMV35S promoter using smaller i/r GUS sequences such GUShp41a (41 bp in the stem spaced by a 9 bp non-GUS sequence; SEQ ID No 10) and GUShp21 (21 bp in the stem spaced by a 6 bp non-GUS sequence, SEQ ID No 11) (Table 2).

TABLE 2
Summary of constructs tested in tobacco

| Constructs | Description |
|---|---|
| pMBW465 | GUShp94 driven by 35S promoter (pART7) |
| pMBW466 | GUShp94 driven by AtU3+136 |
| pMBW468 | GUShp94 driven by AtU3 |
| pMBW470 | GUShp94 driven by At7SL |
| pMBW472 | GUShp94 driven by At7SL+86 |
| pMBW473 | GUShp94 driven by OsU3 |
| pMBW476 | GUShp94 driven by AtU3+3 |
| pMBW477 | GUShp94 driven by AtU3+20 |
| pLMW64 | GUShp94 driven by TomU3 |
| pLMW53 | GUShp41a driven by 35S promoter |
| pLMW58 | GUShp41a driven by AtU3+136 |
| pLMW61 | GUShp21c driven by AtU3+136 |

These constructs were introduced to binary vectors pART27 or pWBVec4a for plant transformation. Two different transgenic tobacco lines expressing GUS, were transformed by all these constructs. A control construct in which the GUShp94 sequence was driven by a 35S promoter (in pART7) was also included.

Leaf tissue from transformed tobacco plantlets on rooting medium was assayed for GUS activity (fluorometric MUG assay) and the results are summarized in Table 3.

The results show that the GUShp94 constructs with AtU3 (pMBW468), At7SL (pMBW470), At7SL+86 (pMBW472), AtU3+3 (pMBW476), AtU3+20 (pMBW477) and TomU3 (pLMW64) promoters all activated silencing of the GUS gene in tobacco. The AtU3, AtU6+20, and TomU3 constructs appeared to perform better than the others. The AtU3+136 construct (pMBW466) did not seem to give significant GUS silencing in tobacco. Also, the OsU3 construct (pMBW473) appeared to confer only a low level of GUS silencing. The PolIII promoter construct pLMW58 (AtU3+136-GUShp41a) gave significant levels of GUS silencing in tobacco whereas the 35S construct pLMW53 (35S-GUShp41a) did not, suggesting that the PolIII promoters are more effective than the PolII promoters in driving the expression of small hairpin RNA.

TABLE 3
MUG assay of tobacco leaf tissue transformed with
constructs listed in Table 2 (5 μg protein)

| Constructs | Untransformed | 465 | 466 | 468 | 470 | 472 | 473 | 476 | 477 | 64 | 53 | 58 | 61 |
|---|---|---|---|---|---|---|---|---|---|---|---|---|---|
| PPGH2 | 40.0 | 2.0 | 17.4 | 2.2 | 8.2 | 5.6 | 11.3 | 8.4 | 5.2 | 7.9 | 55.4 | 20.6 | 6.0 |
| GUS | 51.1 | 21.4 | 34.0 | 12.8 | 10.9 | 5.6 | 17.0 | 8.2 | 3.9 | 6.6 | 53.4 | 17.2 | 19.4 |
| background | 51.0 | 1.8 | 13.9 | 4.5 | 9.2 | 12.8 | 14.9 | 22.9 | 35.5 | 2.9 | 27.0 | 1.4 | 25.8 |
| | 46.8 | 0.6 | 16.0 | 12.92 | 6.9 | 7.8 | 4.2 | 36.5 | 3.7 | 20.9 | | 9.1 | 29.6 |
| | | 0.7 | 24.7 | 6.9 | 12.5 | 9.9 | 15.3 | | 8.4 | 11.0 | | | 24.1 |
| | | 3.5 | 20.2 | 7.1 | | 10.8 | 2.1 | | 10.9 | 9.0 | | | 21.6 |
| | | 23.3 | | | | 19.1 | 17.1 | | 1.8 | 6.7 | | | 25.5 |
| | | | | | | 5.5 | 24.3 | | 32.7 | 3.3 | | | 13.7 |
| | | | | | | 13.3 | 20.5 | | 3.9 | 13.6 | | | 31.3 |
| | | | | | | 13.9 | 20.8 | | | | | | 57.4 |
| | | | | | | | | | | | | | 43.5 |
| | | | | | | | | | | | | | 12.8 |
| PPHG24 | 17.7 | 18.9 | 39.8 | 19.8 | 2.54 | 1.7 | 9.2 | 13.4 | 14.5 | 6.6 | 50.0 | 30.6 | 14.6 |
| GUS | 32.4 | 0.5 | 23.9 | 18.1 | 38.8 | 20.6 | 11.0 | 18.2 | 9.1 | | 48.4 | 9 | 43.9 |
| background | 54.6 | | 15.5 | 4.3 | 15.5 | 5.8 | 16.7 | 4.2 | | | 25.9 | | 26.8 |
| | 18.6 | | 13.8 | 9.3 | 8.2 | | 14.8 | 10.9 | | | | | 8.6 |
| | | | | 5.0 | | | 12.9 | | | | | | |
| | | | | 11.6 | | | 16.5 | | | | | | |
| | | | | | | | 25.8 | | | | | | |

### Example 3

### Testing of the PolIII Promoters in Gene Silencing Constructs Against a GUS Reporter Gene (Arabidopsis thaliana).

Similar constructs as in Example 2 were generated and cloned in pWBVec4a (see Table 4) and were used to transform a transgenic Arabidopsis line, expressing a CaMV35S-GUS gene.

TABLE 4
Summary of constructs tested in Arabidopsis

| Constructs | Description |
|---|---|
| pMBW479 | GUShp94 driven by 35S promoter (pART7) |
| pMBW480 | GUShp94 driven by AtU3+136 |
| pMBW481 | GUShp94 driven by AtU3 |
| pMBW482 | GUShp94 driven by At7SL |
| pMBW483 | GUShp94 driven by At7SL+86 |
| pMBW485 | GUShp94 driven by OsU3 |
| pMBW486 | GUShp94 driven by AtU3+3 |
| pMBW488 | GUShp94 driven by AtU3+20 |
| pLMW62 | GUShp94 driven by TomU3 |
| pLMW56 | GUShp41 driven by 35S promoter |
| pLMW52 | GUShp41 driven by AtU3+136 |
| PLMW60 | GUShp21 driven by AtU3+136 |

Leaf tissues from T1 plants that showed high-levels of resistance to the selective agent PPT were assayed for GUS activity. The MUG assay data are summarized in Table 5.

For the GUShp94 sequence all the U3 and U6 promoter-driven constructs conferred GUS silencing, although the TomU3 and AtU6+20 gave more consistent and better silencing. The two At7SL promoter constructs did not appear to confer significant GUS silencing although a few lines showed moderate silencing, which may be due to T-DNA insertion next to endogenous promoters.

With the GUShp41 sequence, the AtU3+136 construct performed better than the 35S construct in terms of the degree of GUS silencing, again suggesting that PolIII promoters are more effective than PolII promoters for driving expression of small hairpin RNA expression in plants.

TABLE 5
MUG assay of Arabidopsis leaf tissue super-transformed
with constructs listed in Table 4 (5 μg protein)

| Contructs | Untransformed | 479 | 480 | 481 | 482 | 483 | 485 | 486 | 488 | 62 | 56 | 52 | 60 |
|---|---|---|---|---|---|---|---|---|---|---|---|---|---|
| 35S-GUS | 56.1 | 1.53 | 19.9 | 3.11 | 14.1 | 4.25 | 0.30 | 4.50 | 2.19 | 9.58 | 9.30 | 3.63 | 6.43 |
| background | 65.5 | 0.33 | 3.50 | 2.37 | 8.53 | 75.3 | 6.79 | 14.4 | 1.99 | 0 | 6.98 | 1.52 | 55.3 |
| | 69.5 | 75.4 | 15.0 | 14.1 | 6.71 | | 30.2 | 0 | 7.97 | 0 | 7.42 | 3.80 | 55.3 |
| | 45.0 | 8.73 | 2.8 | 25.3 | 35.6 | | 3.88 | | 17.8 | 0 | 9.35 | | 63.1 |
| | 68.4 | 1.50 | 7.0 | 2.90 | 15.6 | | 16.4 | | 10.7 | 15.5 | 6.60 | | 8.07 |
| | | 1.13 | 18.9 | 4.53 | 48.3 | | 9.03 | | 1.08 | | 56.6 | | 29.0 |
| | | | 2.97 | 1.79 | 39.9 | | | | 6.83 | | | | 81.6 |
| | | | 2.48 | | 61.9 | | | | 2.96 | | | | |
| | | | 13.2 | | 94.4 | | | | 47.9 | | | | |
| | | | | | | | | | 4.32 | | | | |
| | | | | | | | | | 2.49 | | | | |
| | | | | | | | | | 10.1 | | | | |

### Example 4

### Testing of the PolIII Promoters in Gene Silencing Constructs Against a GUS Reporter Gene (Oryza sativa).

The constructs pMBW479, pMBW481, pMBW485, pMBW486 and pLMW62 (see Table 4) were super-transformed into rice that expresses a Ubil-GUS-nos gene. GUS staining showed that only pMBW485 (OsU3-GUShp94) and pMBW479 (35S-GUShp94) conferred significant silencing to the resident GUS gene. These results indicate that dicotyledonous type 3 PolIII promoters will not function in monocots.

### Example 5

### Testing of the PolIII Promoters in Gene Silencing Constructs Against Arabidopsis Endogenous Genes.

The Arabidopsis U6-26 construct contains the promoter from −446 to +3 bp (SEQ ID 5) and additional sequences added by PCR creating XhoI sites at each end of the fragment. These were used to clone the PCR product into the SalI site of a pGEM derived plasmid. The insert was excised with NotI and inserted into the pART27 binary vector for plant transformation. The PCR also incorporated a SalI site between the promoter and termination sequences (T8) for insertion of oligonucleotide sequences.

Two genes were targeted, phytoene desaturase (PDS—silencing gives a photobleached phenotype) and phytochrome B (PHYB—silencing gives hypocotyl elongation in white light). For PDS a single target region was chosen, for PHYB, three target regions were used, respectively from the 5′UTR, a region of the coding region conserved between phytochromes and the 3′ UTR. For each target region two oligonucleotides were made, one to make a double stranded section of 21 bp long, the other to make a 42 bp double stranded section. The double stranded oligos were made as two single strands (upper and lower) and annealed to form a double stranded DNA fragment. Overhang sequences were included at the 5′ and 3′ ends to create SalI compatible ends. The oligo sequences are represented in the sequence listing as SEQ ID 12 to 23 for the PHYB constructs and SEQ ID 24 to 27 for PDS constructs.

The PDShp42 constructs gave phenotypes in most of the examined plants. The results are summarized in Table 6. Insertion of the construct with the dsRNA coding region PDS42 under control of the 35S promoter resulted in more plants with no silencing phenotype than the construct with the dsRNA coding region PDS42 under control of the U6 promoter, and plants with a phenotype only showed the weak bleached cotyledon phenotype and no bleaching of the leaves.

TABLE 6
PDS scores (number of T1 seedlings showing phenotype)

| Phenotype | U6+PDS42 | 35S+PDS42 |
|---|---|---|
| No phenotype | 2 | 17 |
| Bleached cotyledons only | 0 | 26 |
| Total bleaching (cotyledons and 1st pair | 43 | 0 |
| of leafs | | |

For the PHYB silencing experiments, most satisfactory silencing results are obtained with the PHYBC42 dsRNA coding region, where most of the plants show more elongation than the controls. Most of the other constructs do not show a phenotype or else only have one or two plants showing a phenotype suggesting that the choice of target sequence may be important.

Hypocotyl lengths of white light grown plants were measured and grouped in 5 mm categories. From the summary of the data in Table 7, it appears that the U6 promoter driven construct is more effective that the CaMV35S promoter driven construct, but the results are not as pronounced as for the above mentioned PDS gene silencing experiments.

Thus, the following conclusion scan be drawn from the experiments:

1) Type 3 Pol III promoters can be used to effectively drive the expression of dsRNA molecules in plant cells.

2) The At U6 promoter seems to be the most effective promoter tested.

3) The monocot PolIIII promoter is functional both in monocotyledonous and dicotyledonous plants, but the dicotyledonous promoters seem not to be functional in monocotyledonous plants.

4) The type III Pol III promoters appear to be more effective than CaMV35S promoter for gene silencing with relatively short hairpin sequences.

TABLE 7
Silencing of PHYB.

| Categories | | | |
|---|---|---|---|
| Hypocotyl length | | | |
| (cm) | WT | 35S-PHYbC42 | U6-PHYbC42 |
| 0.1-0.5 | | | |
| 0.6-1.0 | 13 | 1 | 2 |
| 1.1-1.5 | 10 | 2 | 5 |
| 1.6-2.0 | 3 | 5 | 10 |
| 2.1-2.5 | | 6 | 6 |
| 2.6-3.0 | 5 | | 9 |
| 3.1-3.5 | 3 | 3 | 2 |
| 3.6-4.0 | | 1 | 11 |
| 4.1-4.5 | | | 2 |
| 4.6-5.0 | | 1 | 1 |

### Example 6

### Additional Experiments with Small Hairpin RNA Encoding Constructs

By using a the cloning strategy as outlined FIG. 1, 20 new small hairpin constructs, as summarized in Table 7, were prepared. The predicted small hpRNAs from all of these constructs comprise a 42 bp dsRNA stem (corresponding to the target gene sequences) and a 9-nt loop (non-target sequence). Three target sequences corresponding to different regions of EIN2 (represented in SEQ ID 31 to 33) or GUS (represented in SEQ ID 28 to 30) were selected. These constructs have been used to transform tobacco to assess their efficacy for inducing the silencing of corresponding endogenous (EIN2) or reporter (GUS) genes.

TABLE 7
Summary of additional small hairpin
RNA encoding constructs

| | | Hairpin | | | |
|---|---|---|---|---|---|
| | | sequence (42 | | | Hairpin |
| Name | Promoter | nt stem) | Name | Promoter | sequence |
| pLMW154 | 35S | GUS-A (SEQ | pLMW164 | TomU3 | GUS-A |
| | | ID No 28) | | | (SEQ ID |
| | | | | | No 28) |
| pLMW155 | 35S | GUS-B(SEQ | pLMW165 | TomU3 | GUS-B |
| | | ID No 29) | | | (SEQ ID |
| | | | | | No 29) |
| pLMW156 | 35S | GUS-C(SEQ | pLMW166 | TomU3 | GUS-C |
| | | ID No 30) | | | (SEQ ID |
| | | | | | No 30) |
| pLMW157 | 35S | EIN-A(SEQ ID | pLMW167 | TomU3 | EIN-A |
| | | No 31) | | | (SEQ ID |
| | | | | | No 31) |
| pLMW158 | 35S | EIN-B(SEQ ID | pLMW168 | TomU3 | EIN-B |
| | | No 32) | | | (SEQ ID |
| | | | | | No 32) |
| pLMW159 | AtU3B | GUS-A(SEQ | pLMW169 | AtU6 | GUS-A |
| | | ID No 28) | | | (SEQ ID |
| | | | | | No 28) |
| pLMW160 | AtU3B | GUS-B(SEQ | pLMW170 | AtU6 | GUS-B |
| | | ID No 29) | | | (SEQ ID |
| | | | | | No 29) |
| pLMW161 | AtU3B | GUS-C(SEQ | pLMW171 | AtU6 | GUS-C |
| | | ID No 30) | | | (SEQ ID |
| | | | | | No 30) |
| pLMW162 | AtU3B | EIN-A(SEQ ID | pLMW172 | AtU6 | EIN-A |
| | | No 31) | | | (SEQ ID |
| | | | | | No 31) |
| pLMW163 | AtU3B | EIN-B(SEQ ID | pLMW173 | AtU6 | EIN-B |
| | | No 32) | | | (SEQ ID |
| | | | | | No 32) |

Tobacco shoots were assayed for GUS expression, and the result is shown in Table 8. The result shows that

1) most of the small GUS hairpin constructs conferred good GUS silencing; and

2) the PolIII promoter driven constructs pLMW164 and pLMW165 result in more consistent GUS silencing than the 35S promoter driven construct pLMW155.

The tobacco tissue assayed was mostly leaf pieces from small regenerating shoots growing on medium with hygromycin, which usually gives tight selection.

TABLE 8
GUS activity of putatively transformed tobacco shoots

| | | LMW | LMW | LMW | LMW | LMW | LMW | LMW |
|---|---|---|---|---|---|---|---|---|
| Construct | Untransformed | 155 | 156 | 164 | 165 | 166 | 169 | 170 |
| MUG assay | 51.2 | 7.14 | 0.76 | 2.16 | 12.4 | 1.27 | 0.94 | 1.75 |
| reading | 37.0 | 20.2 | | 0.40 | 1.55 | 68.3 | 1.45 | 2.40 |
| | 52.2 | 0.16 | | 1.41 | 2.22 | 27.6 | 1.83 | 25.32 |
| | 44.32 | 8.98 | | | 0.81 | 7.07 | 7.74 | 0.70 |
| | | 3.04 | | | 2.31 | | 37.5 | 52.7 |
| | | 0.17 | | | 1.19 | | 3.35 | 26.0 |
| | | 70.3 | | | 1.24 | | 25.3 | 7.65 |
| | | 74.3 | | | | | 85.3 | 1.31 |
| | | 81.9 | | | | | | 2.00 |
| | | | | | | | | 9.18 |
| | | | | | | | | 0.69 |
| | | | | | | | | 19.6 |
| | | | | | | | | 1.37 |
| | | | | | | | | 1.75 |
| | | | | | | | | 0.92 |
| | | | | | | | | 35.3 |

### REFERENCES

Bourque and Folk (1992) Plant Mol. Biol. 19: 641-647

Brummelkamp et al. (2002) Science 296: 550-553

Elbashir et al. (2001) Nature 411: 494-498

Fire et al. (1998) Nature 391: 806-811

Hamilton et al. (1998) Plant J. 15: 737-746

Miyagishi et al. (2002) Nature Biotechnology 20: 497-499

Needleman and Wunsch (1970) J. Mol. Biol. 48: 443-453

Paddison et al. (2002) Genes & Development 16: 948-958

Paul et al. (2002) Nature Biotechnology 20: 505-508

Sook Lee et al. (2002) Nature Biotechnology 20: 500-505

Sui et al. (2002) Proc. Natl. Acad. Sci. USA 99: 5515-5520

Waterhouse et al (1998) Proc. Natl. Acad. Sci. USA 95: 13959-13964

Yukawa et al. (2002) Plant Mol. Biol. 50: 713-723

1. A method for reducing the expression of a gene of interest in a plant cell, comprising the following steps:
a. providing a chimeric gene to said plant cell, said chimeric gene comprising the following operably linked DNA fragments:
i. a promoter recognized by a DNA dependent RNA polymerase III of said plant cell characterized in that said promoter is a promoter of type III comprising all cis-acting promoter elements which interact with said DNA dependent RNA polymerase III;
ii. a DNA fragment which, when transcribed, yields an RNA molecule, said RNA molecule comprising a sense and antisense nucleotide sequence,
(1) said sense nucleotide sequence comprising about 19 contiguous nucleotides having about 90 to about 100% sequence identity to a nucleotide sequence of about 19 contiguous nucleotide sequences from the RNA transcribed from said gene of interest;
(2) said antisense nucleotide sequence comprising about 19 contiguous nucleotides having about 90 to 100% sequence identity to the complement of a nucleotide sequence of about 19 contiguous nucleotide sequence of said sense sequence;
wherein said sense and antisense nucleotide sequence are capable of forming a double stranded RNA of about 19 to about 200 nucleotides in length; and
iii. an oligo dT stretch comprising at least 4 consecutive T-residues; and
b. identifying plant cells wherein said expression of said gene of interest is reduced when compared to the expression of said gene of interest in plant cells which do not comprise said chimeric gene.

2. The method according to claim 1, wherein said promoter is a type 3 POLIII promoters selected from the promoter of a plant gene encoding U6snRNA, the promoter of a plant gene encoding U3snRNA or the promoter of a plant gene encoding 7SL RNA.

3. The method according to claim 1, wherein said promoter comprise a nucleotide sequence selected from the nucleotide sequences of SEQ ID No. 1 from the nucleotide at position 7 to the nucleotide at position 322, SEQ ID No.2 from the nucleotide at position 7 to the nucleotide at position 408, SEQ ID No.3 from the nucleotide at position 7 to the nucleotide at position 313, SEQ ID No.4 from the nucleotide at position 7 to the nucleotide at position 446, SEQ ID No.5 from the nucleotide at position 7 to the nucleotide at position 436, SEQ ID No.6 from the nucleotide at position 7 to the nucleotide at position 468, SEQ ID No.7 from the nucleotide at position 7 to the nucleotide at position 384 or SEQ ID No.8 from the nucleotide at position 7 to the nucleotide at position 421.

4. The method according to any one of claims 1 to 3 claim 1, wherein said plant cell is a dicotyledonous plant cell and said promoter is derived from a dicotyledonous or monocotyledonous plant or plant cell.

5. The method according to any one of claims 1 to 3 claim 1, wherein said plant cell is a monocotyledonous plant cell, and said promoter is derived from a monocotyledonous plant or plant cell.

6. The method according to any one of claims 1 to 3 claim 1 wherein said promoter is endogenous to said plant cell.

7. The method according to any one of claims 1 to 6 claim 1, wherein said gene of interest is a transgene.

8. The method according to any one of claims 1 to 6 claim 1, wherein said gene of interest is an endogenous gene.

9. The method according to any one of claims 1 to 8 claim 1, wherein said plant cell is comprised within a plant.

10. A chimeric gene as described in any of claims 1 to 9 claim 1.

11. A plant cell comprising a chimeric gene according to claim 10.

12. A plant comprising within its plant cells a chimeric gene according to claim 10.


SEQUENCE LISTING (33 sequences)

SEQ ID NO: 1 - DNA, 341 bp, Artificial Sequence
  sequence of the promoter of the 7SL-2 gene of Arabidopsis thaliana var. Landsberg erecta
ctcgagatgttgttgttaccagaaagtaaataaatgttcaatctctgatgttctcaagtaagtgagttttattgggaataatattaactcatgttcttctgcatttgattcctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttgttacgtcgactttttttctcgag

SEQ ID NO: 2 - DNA, 429 bp, Artificial Sequence
  sequence of the promoter of the 7SL-2 gene of Arabidopsis thaliana var. Landsberg erecta including 86 bases downstream of the transcription initiation site.
ctcgagatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcttttgtgtttgtttatatattacacgaaccgatctttggtacgtcgagctaagtaacatgagcttgtaacccatgtggggacattaagatggtggaacactggctcgggtccacgggccggttctgttgtcgactttttttttctcgag

SEQ ID NO: 3 - DNA, 334 bp, Artificial Sequence
  sequence of the promoter of the U3 snRNA of Arabidopsis thaliana var. Landsberg erecta
gaattcttatgcagcctgtgatggataactgaatcaaacaaatggcgtctgggtttaagaagatctgttttggctatgttggacgaaacaagtgaacttttaggatcaacttcagtttatatatggagcttatatcgagcaataagataagtgggctttttatgtaatttaatgggctatcgtccatagattcactaatacccatgcccagtacccatgtatgcgtttcatataagctcctaatttctcccacatcgctcaaatctaaacaaatcttgttgtatatataacactgagggagcaacattggtcacgatcgtttttttttgaattc

SEQ ID NO: 4 - DNA, 467 bp, Artificial Sequence
  sequence of the promoter of the U3 snRNA gene of Arabidopsis thaliana var. Landsberg erecta including 136 bases downstream of the transcription initiation site.
gaattcttatgcagcctgtgatggataactgaatcaaacaaatggcgtctgggtttaagaagatctgttttggctatgttggacgaaacaagtgaacttttaggatcaacttcagtttatatatggagcttatatcgagcaataagataagtgggctttttatgtaatttaatgggctatcgtccatagattcactaatacccatgcccagtacccatgtatgcgtttcatataagctcctaatttctcccacatcgctcaaatctaaacaaatcttgttgtatatataacactgagggagcaacattggtcacgaccttacttgaacaggatctgttctataggctcgtacctctgtttccttgatttctcaagagacaggcccttaaccctggttgatgaaccatgaccgtgcggctagagcgtgattgacggctacgatcgtcctcgagtttttttttgaattc

SEQ ID NO: 5 - DNA, 456 bp, Artificial Sequence
  sequence of the promoter of the U6 snRNA gene of Arabidopsis thaliana var. Landsberg erecta including 3 bases downstream of the transcription initiation site
ctcgagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgacttttttttgagctc

SEQ ID NO: 6 - DNA, 488 bp, Artificial Sequence
  sequence of the promoter of the U3 snRNA gene of Arabidopsis thaliana var. Landsberg erecta including 20 bases downstream of the transcription initiation site
ctcgagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcccttcggggacatccgatcgttttttttctcgag

SEQ ID NO: 7 - DNA, 405 bp, Artificial Sequence
  sequence of the promoter of the U3 snRNA of rice (Oryza sativa Indica IR36)
gaattcaagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcacgatcgtttttttttgaattc

SEQ ID NO: 8 - DNA, 442 bp, Artificial Sequence
  sequence of the promoter of the U3 snRNA of tomato (a garden variety with small gourd-shaped yellow fruit)
gaattctgagagcattgtgtggcgttcctctgaattacttactgtcactttgattggagccattattttcagactctactgaagattgaattgaatgagaaactatgaaactttacaagtgaattattatggagttcatggcaactgctatggagtttttcctactgggaattggaacggtttctacgaaattaactgtccacacgttaaaaatataaattaatgcgtaattgttattttttctataacaaataaaaaactgaaatacgacataaattttattactttaattgcactttagccttagagatattgcgttgtagtcggcgtaggtgtgtcaggggccaatatattgttcccacatcggcagtgcagcacataaactctagcgttataagaatctatccactatcaacggtcacgatcgtttttttttgaattc

SEQ ID NO: 9 - DNA, 295 bp, Artificial Sequence
  sequence of the dsRNA encoding region of 94bp for silencing expression of the GUS gene (GUShp94)
gtcgacgatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggatccagacgcgtggttacagtcttgcgcgacatgcgtcaccacggtgatatcgtccacccaggtgttcggcgtggtgtagagcattacgctgcgatcgtcgac

SEQ ID NO: 10 - DNA, 93 bp, Artificial Sequence
  sequence of the dsRNA encoding region of 41 bp for silencing expression of the GUS gene (GUShp41)
gtcgactgggcagatgaacatggcatcgtggtgattgatgaatgcgagaacttcatcaatcaccacgatgccatgttcatctgcccagtcgac

SEQ ID NO: 11 - DNA, 50 bp, Artificial Sequence
  sequence of the dsRNA encoding region of 21 bp for silencing expression of the GUS gene (GUShp21)
gtcgactgggcagatgaacatgtacgatcatgttcatctgcccagtcgac

SEQ ID NO: 12 - DNA, 94 bp, Artificial Sequence
  sequence of the dsRNA encoding region of 42 bp for silencing expression of the PHYB gene, derived from the 5′ end of PHYB (PHYB5hp 42)-upper strand
tcgacggagtcgggggtagtggcggtggccgtggcggtggccgtggaggaggccacggccaccgccacggccaccgccactacccccgactccg

SEQ ID NO: 13 - DNA, 94 bp, Artificial Sequence
  sequence of the dsRNA encoding region of 42 bp for silencing expression of the PHYB gene, derived from the 5′ end of PHYB (PHYB5hp 42)-lower strand
tcgacggagtcgggggtagtggcggtggccgtggcggtggccgtggcctcctccacggccaccgccacggccaccgccactacccccgactccg

SEQ ID NO: 14 - DNA, 52 bp, Artificial Sequence
  sequence of the dsRNA encoding region of 21 bp for silencing expression of the PHYB gene, derived from the 5′ end of PHYB (PHYB5hp 21)-upper strand
tcgacggagtcgggggtagtggcggaggaggccgccactacccccgactccg

SEQ ID NO: 15 - DNA, 52 bp, Artificial Sequence
  sequence of the dsRNA encoding region of 21 bp for silencing expression of the PHYB gene, derived from the 5′ end of PHYB (PHYB5hp 21)-lower strand
tcgacggagtcgggggtagtggcggcctcctccgccactacccccgactccg

SEQ ID NO: 16 - DNA, 94 bp, Artificial Sequence
  sequence of the dsRNA encoding region of 42 bp for silencing expression of the PHYB gene, derived from the center of PHYB (PHYBChp 42)-upper strand
tcgatggatggtgtggttcagccatgtagggatatggcgggggaacaggagggttcccccgccatatccctacatggctgaaccacaccatcca

SEQ ID NO: 17 - DNA, 94 bp, Artificial Sequence
  sequence of the dsRNA encoding region of 42 bp for silencing expression of the PHYB gene, derived from the center of PHYB (PHYBChp 42)-lower strand
tcgatggatggtgtggttcagccatgtagggatatggcgggggaaccctcctgttcccccgccatatccctacatggctgaaccacaccatcca

SEQ ID NO: 18 - DNA, 52 bp, Artificial Sequence
  sequence of the dsRNA encoding region of 21 bp for silencing expression of the PHYB gene, derived from the center of PHYB (PHYBChp 21)-upper strand
tcgatggatggtgtggttcagccataggaggatggctgaaccacacctccaa

SEQ ID NO: 19 - DNA, 52 bp, Artificial Sequence
  sequence of the dsRNA encoding region of 21 bp for silencing expression of the PHYB gene, derived from the center of PHYB (PHYBChp 21)-lower strand
tcgatggatggtgtggttcagccatcctcctatggctgaaccacaccatcca

SEQ ID NO: 20 - DNA, 94 bp, Artificial Sequence
  sequence of the dsRNA encoding region of 42 bp for silencing expression of the PHYB gene, derived from the 3′ end of PHYB (PHYB3hp 42)-upper strand
tcgacattgtcaactgctagtggaagtggtgacatgatgctgatgaaggaggtcatcagcatcatgtcaccacttccactagcagttgacaatg

SEQ ID NO: 21 - DNA, 94 bp, Artificial Sequence
  sequence of the dsRNA encoding region of 42 bp for silencing expression of the PHYB gene, derived from the 3′ end of PHYB (PHYB3hp 42)-lower strand
tcgacattgtcaactgctagtggaagtggtgacatgatgctgatgacctccttcatcagcatcatgtcaccacttccactagcagttgacaatg

SEQ ID NO: 22 - DNA, 52 bp, Artificial Sequence
  sequence of the dsRNA encoding region of 21 bp for silencing expression of the PHYB gene, derived from the 3′ end of PHYB (PHYB3hp 21)-upper strand
tcgacattgtcaactgctagtggaaaggaggttccactagcagttgacaatg

SEQ ID NO: 23 - DNA, 52 bp, Artificial Sequence
  sequence of the dsRNA encoding region of 21 bp for silencing expression of the PHYB gene, derived from the 3′ end of PHYB (PHYB3hp 21)-lower strand
tcgacattgtcaactgctagtggaacctcctttccactagcagttgacaatg

SEQ ID NO: 24 - DNA, 94 bp, Artificial Sequence
  sequence of the dsRNA encoding region of 42 bp for silencing expression of the PDS gene (PDS42)-upper strand
tcgacttaacttgtaaggaatattacgatcctaaccggtcaatgctaggaggagcattgaccggttaggatcgtaatattccttacaagttaag

SEQ ID NO: 25 - DNA, 94 bp, Artificial Sequence
  sequence of the dsRNA encoding region of 42 bp for silencing expression of the PDS gene (PDS42)-lower strand
tcgacttaacttgtaaggaatattacgatcctaaccggtcaatgctcctcctagcattgaccggttaggatcgtaatattccttacaagttaag

SEQ ID NO: 26 - DNA, 52 bp, Artificial Sequence
  sequence of the dsRNA encoding region of 21 bp for silencing expression of the PDS gene (PDS21)-upper strand
tcgacttaacttgtaaggaatattaaggaggtaatattccttacaagttaag

SEQ ID NO: 27 - DNA, 52 bp, Artificial Sequence
  sequence of the dsRNA encoding region of 21 bp for silencing expression of the PDS gene (PDS21)-lower strand
tcgacttaacttgtaaggaatattacctccttaatattccttacaagttaag

SEQ ID NO: 28 - DNA, 115 bp, Artificial sequence
  small hairpin RNA coding region (GUS_A)
gtcgacgatcgtgcggtcactcattacggcaaagtgtgggtcaataatcaggagttccttcttcctgattattgacccacactttgccgtaatgagtgaccgcagtcgacgatcg

SEQ ID NO: 29 - DNA, 112 bp, Artificial sequence
  small hairpin RNA coding region (GUS_B)
gtcgacgatcgtcatgaagatgcggacttgcgtggcaaaggattcgataagttccttctttatcgaatcctttgccacgcaagtccgcatcttcatgacgagtcgacgatcg

SEQ ID NO: 30 - DNA, 115 bp, Artificial sequence
  small hairpin RNA coding region (GUS_C)
gtcgacgatcgtgcgacctcgcaaggcatattgcgcgttggcggtaacaagaagttccttctttcttgttaccgccaacgcgcaatatgccttgcgaggtcgcagtcgacgatcg

SEQ ID NO: 31 - DNA, 115 bp, Artificial sequence
  small hairpin RNA coding region (EIN_A)
gtcgacgatcgcatcttatgccaatatgttgcagctcgcataagcgttgtgacgttccttctgtcacaacgcttatgcgagctgcaacatattggcataagatggtcgacgatcg

SEQ ID NO: 32 - DNA, 112 bp, Artificial sequence
  small hairpin RNA coding region (EIN_B)
gtcgacgatcggcaggcctggtattacttctctatgtttctggcgtcttggttccttctcaagacgccagaaacatagagaagtaataccaggcctgccgagtcgacgatcg

SEQ ID NO: 33 - DNA, 115 bp, Artificial sequence
  small hairpin RNA coding region (EIN_C)
gtcgacgatcgcatagctgtttcctgtgtgaaattggtatccgctcacaattcgttccttctgaattgtgagcggataccaatttcacacaggaaacagctatggtcgacgatcg